(19) United States
(12) Patent Application Publication
(10) Pub. No.: US 2009/0187147 A1
(43) Pub. Date: Jul. 23, 2009

(54) APPARATUS AND METHOD FOR ACHIEVING MICROPUNCTURE

(75) Inventors: Paul A. Kurth, Santa Barbara, CA (US); Andrew W. Armour, Swarthmore, PA (US)

Correspondence Address: Law Offices of Daniel L. Dawes, 5200 Warner Blvd, Ste. 106, Huntington Beach, CA, 92649, US

(73) Assignee: Pressure Products Medical Supplies, Inc., Santa Barbara, CA (US)

(21) Appl. No.: 12/017,766

(22) Filed: Jan. 22, 2008

(51) Int. Cl. A61M 25/09

(52) U.S. Cl. 604/161

(57) ABSTRACT

A method comprises the steps of inserting a microneedle or micropuncture device into a body cavity to achieve a micropuncture into the cavity while reducing tissue trauma at a puncture site. A stepped or tapered guidewire having a reduced diameter distal portion is telescopically disposed through the inner diameter of the microneedle or Micropuncture device. A proximal portion of the guidewire has a diameter larger than the inner diameter of the microneedle or Micropuncture device and extends to a proximal end of the guidewire. The proximal portion is adapted for guiding a larger diameter instrument into and through the vascular system. The microneedle or micropuncture device is withdrawn from the micropuncture while leaving the distal portion of the guidewire extending through the micropuncture. The microneedle or micropuncture device is removed from the guidewire without removing the microneedle or Micropuncture device over the proximal end of the guidewire. As a result, tissue trauma at the puncture site is substantially reduced.

### BACKGROUND OF THE INVENTION

1. Field of the Invention

The invention relates to the field of micropuncture endovascular devices and methods, and in particular to micropuncture endovascular systems and guidewires for vascular introduction and methods of using the same.

2. Description of the Prior Art

The prior art methodology used to make a micropuncture is as follows: (1) First the vasculature is punctured with a microneedle. (2)Then placement of a microguidewire through the needle is achieved. (3)Then a coaxial catheter pair is advanced over the microguidewire into the blood vessel. (4) Then the microguidewire is removed and then (5) The inner sleeve of the coaxial catheter pair is removed and discarded. (6) Then a large guidewire is introduced into the vasculature through the remaining outer sleeve (which has an inside diameter large enough to accommodate a large guidewire) of the coaxial catheter pair, (7) Then the retained outer sleeve must be removed. Then the larger introducer or medical device is advanced over the larger guidewire. Seven steps and two guidewires are currently required to place a large guidewire using the micropuncture technique.

The prior art systems require the introduction of two separate guidewires into the vasculature. Each time a guidewire is introduced it carries the risk of damaging the lining of the blood vessel which may result in thrombosis and tissue or organ damage. The prior art systems also require a pair of coaxial catheters to be used in order to deploy a larger diameter guidewire, significantly adding to the number of components and steps required for placement of a large guidewire. The current systems for micropuncture guidewire placement are thus unnecessarily complex and potentially dangerous to the patient.

What is needed is a system that allows for a micropuncture to be achieved while minimizing the risk to the patient. This is achieved by using a (1) removable needle or sleeve for the micropuncture and (2) placing only one guidewire as apposed to two. The invention not only reduces the risk of trauma to the patient's tissues and vasculature but also reduces the number of components and steps required for the overall procedure. Placement of a large guidewire is reduced to placing one guidewire using only two to three procedural steps.

### BRIEF SUMMARY OF THE INVENTION

The illustrated embodiment of the invention is an apparatus and method of using an assembly comprising a longitudinally openable or separable, small diameter, hollow micropuncture device or microneedle for puncture into a vascular or body cavity while minimizing tissue trauma at a puncture site, and a stepped guidewire. The term, “micropuncture device”, shall be used throughout this specification and claims to refer to a needle, microneedle, and/or an assembly of elements which include a microneedle, a needle or an element that functions at least in part as a microneedle or needle having a maximum gauge size of 20, or can accommodate a 0.025 inch maximum sized diameter guidewire. The terms, “non-micropuncture vascular device”, “larger diameter vascular instrument”, “larger medical device”, “large instrument” are defined as a vascular or medical instrument which is too stiff or too large in diameter to be effectively or reliably guided into the vascular system or other body cavity through a micropuncture device or by means of guidewires which are usable in micropuncture devices. The term “microguidewire” refers to a guidewire or a portion of a guidewire that is 0.025 inch diameter or smaller whereas the term “larger guidewire” refers to a guidewire or a portion of a guidewire that is larger than 0.025 inch diameter. The micropuncture device has an inner diameter; and the stepped guidewire has a distal portion for telescopic disposition through the inner diameter of the micropuncture device or needle, and a proximal portion with a diameter larger than the inner diameter of the micropuncture device or needle. The stepped guidewire is provided for guiding a larger diameter vascular instrument. By use of the illustrated embodiment tissue trauma at the puncture site is substantially reduced.

The illustrated embodiment of the invention is thus a method comprising the steps of inserting a microneedle into a body cavity to achieve a micropuncture into the cavity while minimizing tissue trauma at a puncture site. A stepped guidewire, a tapered guidewire, or a two part guidewire such as comprised of a core guidewire and a catheter, or a core guidewire and a coil, where the guidewire has a reduced diameter distal portion, is telescopically disposed through the inner diameter of the microneedle. A proximal portion of the guidewire has a diameter larger than the inner diameter of the microneedle and extends to a proximal end of the guidewire. The proximal portion is adapted for guiding a larger diameter instrument into and through the vascular system. The microneedle is withdrawn from the micropuncture while leaving the distal portion of the guidewire extending through the micropuncture. The microneedle is removed from the guidewire without removing the microneedle over the proximal end of the guidewire. As a result, tissue trauma at the puncture site is substantially reduced.

The illustrated embodiment of the method further comprises the steps of advancing the guidewire into the micropuncture to extend the proximal portion of the guidewire through the micropuncture, and guiding a large diameter instrument over the guidewire through the micropuncture using the proximal portion as a guiding guidewire.

Another embodiment of the method further comprises the steps of advancing the guidewire into the micropuncture to extend only the distal portion of the guidewire through the micropuncture, and guiding a larger diameter instrument telescopically over the guidewire through the micropuncture using the distal portion as a guiding guidewire only through the micropuncture.

In one embodiment the method further comprises the step of advancing the guidewire to extend the proximal portion of the guidewire through the larger diameter instrument in the micropuncture and then into the body cavity to provide a guiding guidewire for the larger diameter instrument to a target site in the body cavity.

The step of separating and removing the micropuncture device or needle comprises longitudinally opening, separating, tearing, splitting, peeling, or otherwise removing the micropuncture device or needle along a slotted section, or section line or lines or a score line or lines, or through a weak material, or a material of molecular orientation defined into or on the micropuncture device or needle. For the purposes of this specification, if an element is termed to be “separable”, it is then capable of being opened, separated or separated from something, torn, split, peeled, taken away from something or otherwise removed.

In one embodiment the body cavity comprises a vascular cavity, and the step of inserting a microneedle into a body cavity comprises steps of making a transdermal micropuncture into a vascular cavity.

The illustrated embodiment of the invention is also an apparatus comprising a longitudinally separable, small diameter, hollow micropuncture device or microneedle for puncture into a vascular cavity while minimizing tissue trauma at a puncture site caused by the puncture device. The micropuncture device has an inner diameter. A stepped or tapered guidewire has a distal portion for telescopic disposition through the inner diameter of the micropuncture device, and a proximal portion having a diameter larger than the inner diameter of the micropuncture device and for guiding a larger diameter vascular instrument. As a result tissue trauma at the puncture site is substantially reduced.

In another embodiment the micropuncture device comprises a micropuncture needle, and an openable or separable sleeve mounted over the needle. The micropuncture device then punctures the body cavity or vasculature and deposits or introduces the openable or separable sleeve in the body cavity. The inner puncturing needle is then removed from the outer sleeve. The microguidewire is then passed through the retained or implanted sleeve until the larger diameter of the proximal portion of the guidewire abuts against the smaller inside diameter of the retained sleeve. The sleeve is then opened or removed from the guidewire by separating, tearing, peeling, splitting or any other means now known or devised in the future. The proximal portion of the guidewire can then be advanced through the micropuncture.

In another embodiment, the needle is slotted or split into two portions, but sealed by a thin shrink wrapped or compressed separable sleeve or tube. A hub attached to the needle may also be similarly slotted or split and sealed by the sleeve. The needle is then opened or separated in the above procedure along the slot or split by tearing or separating the sleeve, which preferably encases the exterior of the needle.

The term “sleeve” is used throughout this specification and refers to a material that can be separated to allow removal of the micropuncture device. The sleeve may embody the entire circumference or diameter, or just cover a small portion of the circumference or diameter so as to provide a seal to minimize the passage of fluid. The sleeve can be in the form of a sealant, gel, film, glue, membrane, extrusion, balloon, or any other means that enables removal of the micropuncture device from the guidewire without removing the micropuncture device over the proximal potion of the guidewire.

In one embodiment the distal portion of the guidewire has a diameter of approximately 0.018-0.021 inch and where the proximal portion has a diameter of approximately 0.035-0.038 inch.

The distal portion is characterized as a microguidewire having a diameter such that penetration of the distal portion into tissue and disposition into a vascular cavity reduces tissue trauma.

The apparatus is used in combination with a larger diameter instrument and the proximal portion of the guidewire is characterized as suitable for providing a guiding force to the larger diameter medical instrument which is telescopically disposed over the proximal portion.

The distal portion is chosen with a size of diameter so that it is extremely flexible and atraumatic but is not optimally configured for guiding a larger medical instrument through the vasculature.

The proximal portion of the guidewire has a diameter such that it guides and steers a larger medical instrument in the vascular system.

The distal portion is preferably about 30 cm long and the length of the proximal portion is preferably about 30 cm but the lengths can be adjusted depending on the length of the micropuncture device or needle and the length of the medical instrument that needs to be introduced into the vasculature.

The transition between the proximal and distal portions of the guidewire may be a sharp step. The transition between the proximal and distal portions may be a gradual taper whose longitudinal length is ten diameters of the proximal portion or more. Less steep tapers or a more gradual step are also contemplated within the scope of the invention.

In yet another characterization of the illustrated embodiment of the invention, it is defined as an apparatus for use in introducing a medical instrument into a vascular system comprising a separable means for producing a micropuncture into the vasculature while reducing tissue trauma at a puncture site, and a first means for maintaining access through the puncture site into the vascular system while reducing tissue trauma. The first means for maintaining access is telescopically disposed within the separable means for producing the micropuncture. A second means is provided for guiding the medical instrument within the vascular system. The second means is disposed into the vascular system through the micropuncture after the separable means is separated and removed from the first means for maintaining access.

The first means guides the medical instrument only through the puncture site and a predefined distance into the vascular system.

The separable means comprises a micropuncture device or microneedle with a longitudinal section line or lines or score line or lines along which the micropuncture device or needle can be manually opened or separated.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a side elevational view of the stepped or tapered guidewire.

FIG. 2 is a side cross sectional view showing the insertion of the needle into the puncture site and the distal portion of the guidewire extending through the needle into the vascular system.

FIG. 3 is a side cross sectional view showing the separation and removal of the needle from the guidewire and the advancement of the proximal portion of the guidewire through the puncture site into the vascular system.

FIG. 4 is a perspective view of an embodiment showing the use of a micropuncture device which is provided as a microneedle having a removable sleeve telescopically disposed over it.

FIG. 5 is a perspective view of an embodiment of a slotted, splittable sleeved needle combined with a hub.

FIG. 5a is an enlarged perspective view of the embodiment of FIG. 5 showing the hub and a single wing for handling.

FIG. 6 is a perspective view of an embodiment of a two coaxial slotted sleeved cannula which are rotatable into and out of alignment both with respect to distal bevel tips and longitudinal slots. FIG. 6 shows the configuration where the slots are not aligned, but the bevel tips are.

FIG. 7 is a perspective view of another embodiment of a needle and hub provided with a single longitudinal slot, which is covered by a thin film sleeve.

FIG. 8 is a perspective view of a hub and sleeve having two compressible wings for opening the slot in the hub.

FIG. 9 is a bottom elevational view of the hub of FIG. 8.

FIG. 10 is a side perspective view of a bifurcated hub incorporating an embodiment of the invention similar to FIGS. 5 and 5a.

FIG. 11 is a side perspective view of a bifurcated hub incorporating an embodiment of the invention having two compressible wings for opening the slot in the hub.

FIG. 12 is a perspective view of a valve membrane used in the hubs of the various embodiments of the invention.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

The invention is directed to a system and method for using a microneedle and stepped guidewire to micropuncture a body cavity or endovascular cavity and dispose a microguidewire therein for subsequent disposition of a larger guidewire and instrument such as an introducer or catheter while reducing tissue trauma at the puncture site and to the vascular system.

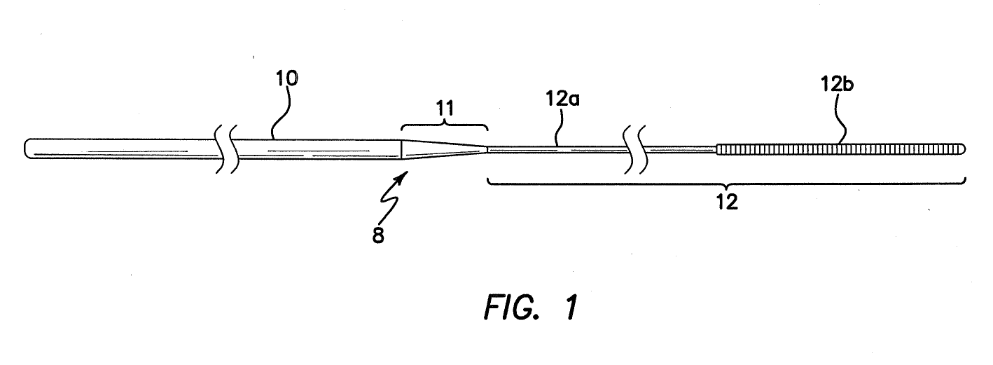

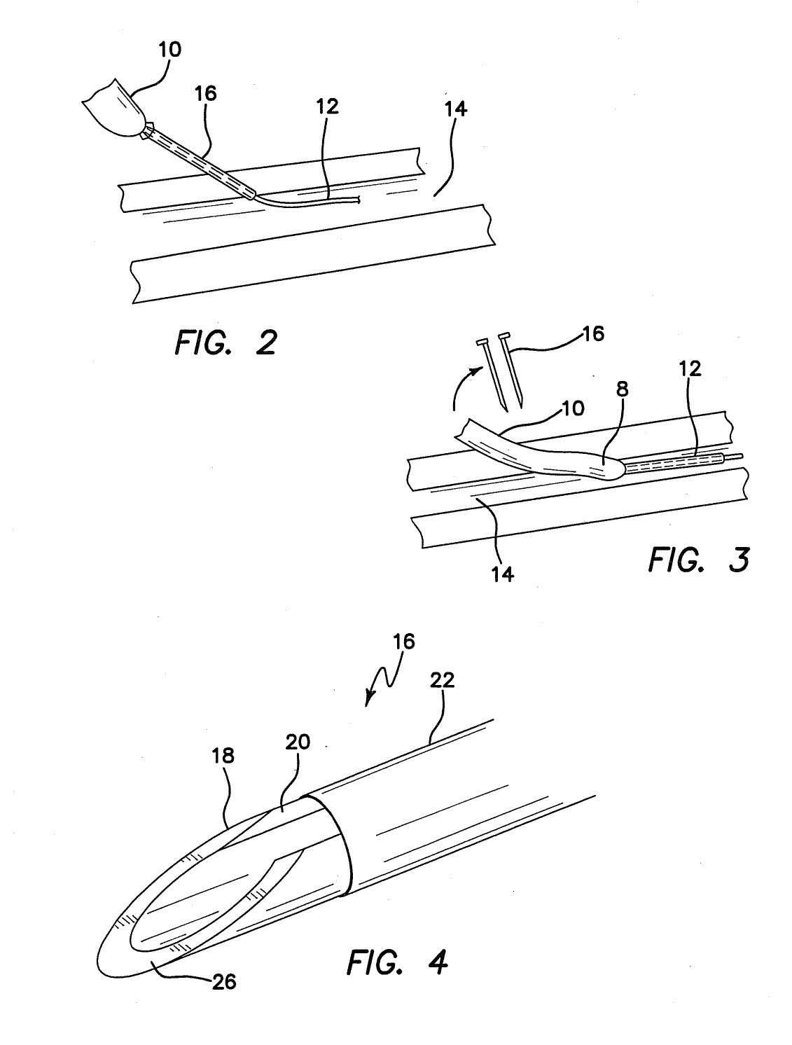

The illustrated embodiment comprises a splittable microneedle 16 as shown in FIG. 2 and a stepped or preferably a steeply tapered guidewire, generally denoted by reference numeral 8, as shown in FIGS. 1-3. As shown in FIG. 1 guidewire 8 has a distal portion 12 comprised of two subportions, namely proximal subportion 12a with a diameter of approximately 0.014 inch and an enlarged distal tip subportion 12b approximately 5 cm in length and approximately 0.018 inch in diameter. In another embodiment portions 12a and 12b may have the same diameters, e.g. 0.18 inch. The two subportions 12a and 12b of distal portion 12 collectively have a length of approximately 29.5 cm. A proximal portion 10 has a diameter of approximately 0.038 inch and a length of approximately 29.5 cm. Portions 10 and 12 of guidewire 8 are joined by a tapered or conical transition 11 of approximately 1 cm length which smoothly transitions between the two differing diameters of portions 10 and 12, but is determined according to ease of manufacturing. It is to be understood that other diameters and ranges can be substituted with equivalency without departing from the scope of the invention. The reduction in diameter of distal portion 12 relative to proximal portion 10 can be achieved by any means known in the art, such as grinding, etching, extruding, cold molding, winding, coiling, welding, soldering, or hot molding distal portion 12 down to its final diameter, or conversely by adding a sleeve, coil, or extrusion to enlarge the proximal portion 10 or both.

The preferred embodiment for the guidewire 8 is comprised of a centerless ground nitinol core guidewire with the proximal portion formed by a coil joined to the nitinol guidewire having an outer diameter of 0.038 inch over a 30 cm length with an intermediate portion consisting of a 1 cm long taper to a 30 cm long distal portion which is comprised of an 0.018 inch diameter nitinol mandrel guidewire from Lake Region Manufacturing Inc. in Chaska, Minn.

Another embodiment for the guidewire 8 is comprised of an 0.018 inch diameter nitinol mandrel guidewire from Lake Region Manufacturing Inc. in Chaska, Minn. that is about 60 cm long. A PEEK (polyetheretherketone, or any other stiff material) catheter (not shown) from Zeus Industrial Products Inc. in Orangeburg, S.C. can be bonded or heat shrunk to the proximal portion 12 of the guidewire 8 that it has an 0.032-0.038 inch outer diameter to provide the stiffness needed. This catheter may also be filled with different fillers (glass, talc, etc.) to change the stiffness of the proximal portion 12.

However, the functional requirement of distal portion 12 is that it be characterized as a microguidewire or have a diameter such that penetration of portion 12 into tissue and disposition into a vascular cavity reduces tissue trauma. The functional requirement of proximal portion 10 is that it be characterized as suitable for providing a guiding force to a larger diameter medical instrument, such as an introducer or catheter, which is telescopically disposed over portion 10. Thus, distal portion 12 is chosen with a size of diameter so that, when it is made of stainless steel, nitinol or other suitable material, which is flexible kink resistant and atraumatic but is not optimally configured to be used to guide a larger medical instrument in the vascular system. In the illustrated embodiment guidewire 8 is constructed on a NiTi core and overlaid in portion 12b with a sheathing made of a stainless steel coil. By the same token distal portion 12 causes a very little disturbance or tissue trauma through the small puncture wound through the skin and vascular wall provided by microneedle 16 through which portion 12 is led as shown in FIG. 2. The diameter of proximal portion 10 is such that it easily carries or guides a larger medical instrument and can be practically steered in the vascular system. The distal more flexible portion of the guidewire can aid in the steering of the more proximal, less flexible, portion of the guidewire.

The stepped guidewire 8 need not have a sharp step, but transitions its diameter in a reasonably short distance as suggested by FIG. 1. A transition 11 between portions 10 and 12 which extends more than one or two diameters of the guidewire 10 is still contemplated within the scope of the invention. As shown in the example of FIG. 1 the length of transition 11 is about 10 times the diameter of adjacent subportion 12a.

As shown in FIG. 2 proximal portion 12 is telescopically disposed through a break-away micropuncture device or needle 16 which is used to achieve a micropuncture into a vascular cavity or vessel 14. The outer diameter of microneedle 16 is preferably in the range of 0.028 to 0.035 inch. The method of use with a break-away microneedle 16 is as follows. A small diameter microneedle 16 is used to puncture and gain entry into vessel 14. The distal portion 12 of the guidewire 8 is inserted into the small diameter microneedle 16 and lead into the vessel 14 as shown in FIG. 2. Proximal portion 10 has a diameter which is too large to be inserted into the microneedle 16 and will limit the extent to which guidewire 8 as a whole can be advanced into microneedle 16. The distal portion 12 of the guidewire is characterized a microguidewire and is too thin and flexible to serve optimally to guide an introducer or any other instrument, in that a larger instrument, such as introducer (not shown), when placed over proximal portion 10 will tend to travel in whatever direction the introducer is inclined to go and carry guidewire portion 12 with it instead of tracking over the guidewire. However, guidewire portion 10 has sufficient guiding properties and strength to serve as an optimal guidewire for a larger instrument into the vessel 14 at the puncture site and through the vasculature.

Once the distal portion of the guidewire is positioned in the vascular cavity 14, microneedle 16 is split, separated or broken apart as per its design, and removed from guidewire 8 leaving the distal portion 12 through the puncture site. The means by which microneedle or micropuncture device 16 can be removed from guidewire 8 without pulling it over the proximal end of the guidewire 8 is not a limiting feature of the invention. Any construction by which microneedle or micropuncture device or a micropuncture device 16 can be separated or partially opened in order to be removed from the guidewire without having to be removed from the proximal end of the guidewire can be employed. In the illustrated embodiment a pair of diametrically opposed score lines 18 in FIG. 2 are machined or molded into a metallic microneedle 16, which allows it to be longitudinally split apart.

The guidewire 8 is then advanced into vessel 14 so that the proximal portion 10 smoothly enters the vessel 14 through the puncture site and is manipulated as per conventional methods to a vascular position as desired. An introducer or other instrument can then be telescoped over proximal portion 10 to the desired position. The extension of guidewire 8 provided by distal portion does not interfere with the use of proximal portion 10 as a guidewire, but may aid in steering the proximal portion of the guidewire through a tortuous vasculature. As a result the proximal portion of the guidewire may effectively steer a larger and stiffer medical device through complex vasculature while reducing initial tissue trauma upon puncturing the vasculature

The result is that a very small microneedle or micropuncture device 16 can be used to create a micropuncture with substantially less tissue trauma and risk to neighboring tissues at the site, less discomfort to the patient and with greater ease of placement than is the case with a needle or puncturing device which is large enough to accommodate the proximal portion 10 through it. Still when an introducer or other instrument needs to be guided over the guidewire 8 to the target site, this is possible by means of conventional manipulation of the proximal portion of the guidewire.

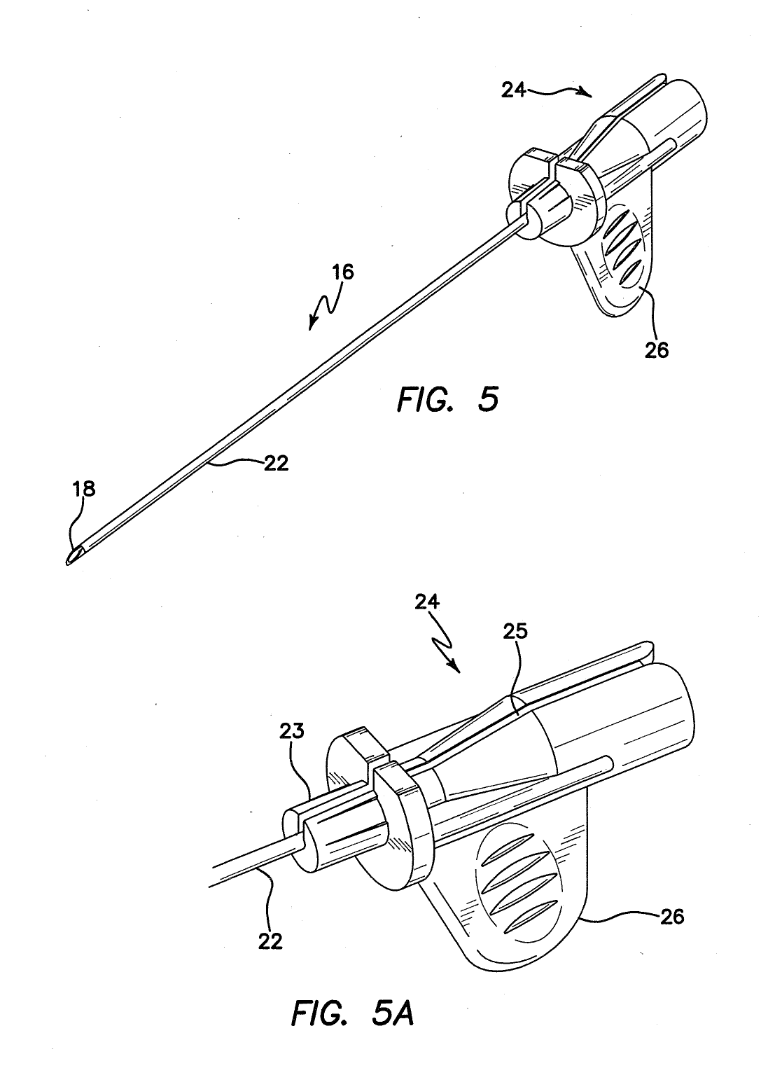

A preferred embodiment for the needle 16 shown in FIGS. 4 and 5 is comprised of a 21 gauge regular walled cannula 18 (0.032″ OD×0.020″ ID) sold by K-Tube Corporation in Poway, Calif. A 0.020 inch wide slot 20 is defined, machined or formed along the longitudinal length of the cannula 18. The slot 20 is then filled and/or coated so that it does not leak. A heat shrink sleeve 22 works well and is made from Pebax®, polytetrafluoroethylene (PTFE) or Teflon®, fluorinated ethylene propylene (FEP) or polyethylene terephthalate (PET). The preferred embodiment uses a thin 0.00025 inch thick walled PET heat shrink tube from Advanced Polymers in Salem, N.H. The proximal portion of the cannula 18 is molded with a slotted hub 24 shown in FIG. 5, and the distal portion of the cannula 18 is ground with a needle bevel 26 as shown best in FIG. 4. In the illustrated embodiment, the distal (0.018″ diameter) portion 12 of the guidewire 8 is placed through the needle 16. The needle hub 24 is held with a single handle or wing 26, and the needle 16 is simply pulled away from the distal portion 12 of the guidewire 8. The guidewire 8 cuts through or tears the thin sleeve 22. A slit (not shown) can be provided in the sleeve 22 to ease the start of the tear as well if needed. As best shown in the enlargement of FIG. 5a a slot 23 is defined in hub 24 through which guidewire 8 may be pulled. In the embodiment of FIG. 5a, slot 23 extends through the entire longitudinal length of hub 24, although this is not to preclude alternative structures where a portion of open slot 23 may not extend the entire length of hub 24. In the proximal portion of hub 24 in the embodiment of FIG. 5a, the slot 23 is open. However, upstream from an internal sealing valve (not shown) in hub 24 slot 23 is closed or sealed with a filler material 25 so that it is fluid-tight. Nevertheless, material 25 filling the more distal portion of slot 23 in hub 24 is soft enough to allow guidewire 8 to be manually pulled therethrough. In another embodiment slot 23 may be closed or fluid-tight along all or a portion of its length and instead of filling by material 25 may be defined by a thinning of the wall thickness or other structural means which will allow guidewire 8 to be pulled through the closed portions of slot 23. For example, instead of a filler material 25, slot 23 may be partially or wholly covered by a shrink-fit, fluid-tight thin sleeve similar to that shown in FIG. 4. The proximal portion 10 of the guidewire 8 may then be advanced into the vasculature. The nitinol core minimizes deformation or kinking of the guidewire 8 when the needle 16 is removed.

Another embodiment of hub 24 is shown in the perspective view of FIG. 8, which is similar that shown in FIGS. 5 and 5a, except that hub 24 of FIG. 8 is provided with two wings 26a and 26b extending from the body 27 of hub 24 and forming a dihedral handle. When wings 26a and 26b are squeezed together, hub 24 is opened along slot 23, which again is provided partially or wholly with a sealed, thin wall along its longitudinal length. Again, the closed portion of slot 23 may be closed by a friable sleeve, an integrally formed friable thin wall of hub 24 which extends across slot 23, a friable filler material 25 or equivalent means for temporarily closing slot 23. In the embodiment of FIG. 8, whatever means closes slot 23 need not be tearable by guidewire 8, but need only be splittable or separable when hub body 27 is flexed and slot 23 stretched apart by compression of wings 26a and 26b together. In this embodiment, slot 23 may be closed simply by resilient compression of the slot 23, which is temporarily opened by flexing hub body 27 when compressing wings 26a and 26b together.

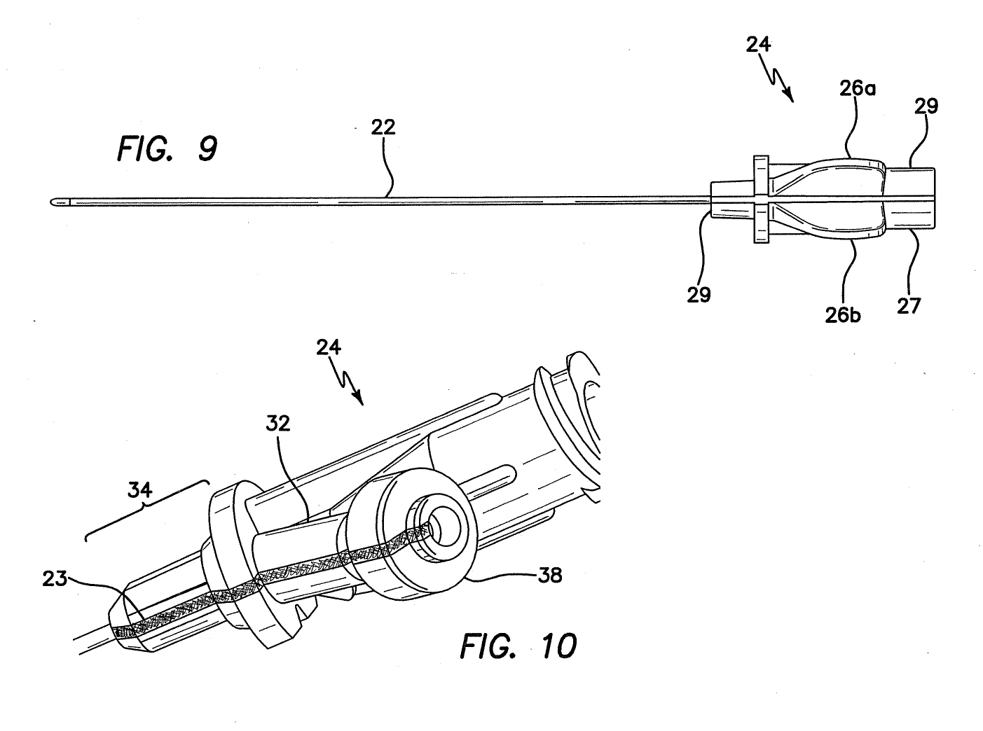

FIG. 9 is a bottom elevational view of the embodiment of FIG. 8. A diametrically opposing groove 29 longitudinally extends along the bottom of hub body 27 opposing slot 23. Wings 26a and 26b attach to body 27 on opposing portions of hub 24 on opposite sides of groove 29. Groove 29 defines a line of flexure or a longitudinal hinge along which hub 24 flexes when wings 26a and 26b are compressed together, thereby assisting in the ease of opening slot 23 when more rigid plastics are utilized for hub body 27. Thus, the material properties chosen for hub 24 will dictate the depth of groove 29 and whether or not a groove 29 is even needed or advantageous.

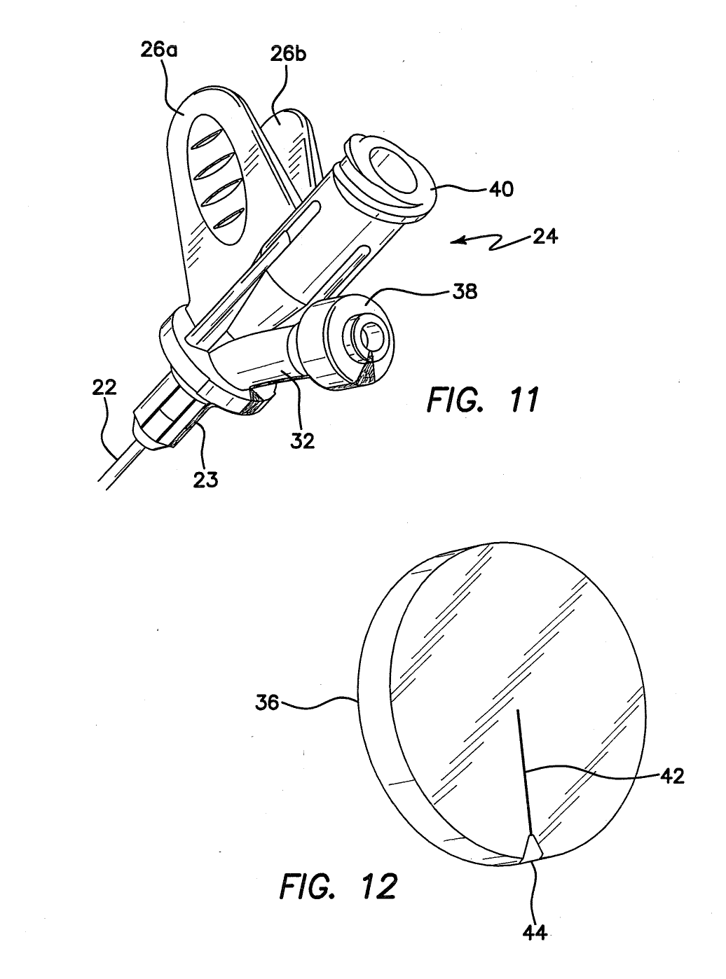

FIG. 10 is a side perspective view of an embodiment of the invention which has been implemented in a bifurcated hub 24 having a second port 32. Slot 23 extends from distal portion 34 and continuously along the longitudinal length of side port 32 including any hubs on port 32. Slot 23 is closed in any one of the modes described above in connection with any one of the embodiments described above. The embodiment illustrated in FIG. 10 depicts the sealed slot design for removing the guidewire 8, and FIG. 11 is a side perspective view of an embodiment of the invention, which depicts a two wing design with a thin wall section that tears/breaks open when the wings 26a and 26b are squeezed together as described in connection with FIG. 9. The bifurcated hub 24 in FIGS. 10 and 11 includes a hemostatic valve membrane 36, and a cap 38 for holding the membrane 36. The bifurcated design of FIGS. 10-12 allows the micropuncture device to be connected to a syringe and introduced in the standard fashion. Once the vessel has been accessed by the needle and blood aspirated into the syringe, the guidewire 8 may be introduced through the bifurcation port 32 and through the hemostatic membrane 36 while the syringe stays attached to a straight Luer fitting 40. This prevents blood from leaking out of the system or air from entering. The stepped guidewire 8 is removed from the hub 24 by tearing through slot 23 formed in the hub 24.

FIG. 12 is perspective diagram of membrane 36 showing a weakened line (a partial cut into membrane 36) or cut 42 (a full cut through membrane 36) that enables the guidewire 8 to cut or pass through membrane 36. A radial section or slit 44 may be provided at the radial extremity of line 42 to facilitate removal of the guidewire 8 therethrough. The membrane 36 is molded from a low durometer (20A-50A) silicone rubber such as Elastosil LR3003/30 from Wacker Silicones Division in Adrian, Mich.

A larger medical device may be guided over guidewire 8 while distal portion 12 occupies the puncture pathway into the body cavity after microneedle 16 is removed from the puncture pathway, or guidewire 8 may be advanced first into the puncture pathway so that proximal portion 10 occupies the puncture pathway when the medical device is guided over guidewire 8. The latter method is preferred in that in many instances the use of the thicker proximal portion 10 of guidewire 8 will be necessary in order to avoid guidewire 8 from being backed out of the body cavity while the medical device is inserted, and/or the tracking of the medical device into the vasculature will be required in a manner that cannot be provided by the lighter, thinner and more flexible distal portion 12 of guidewire 8.

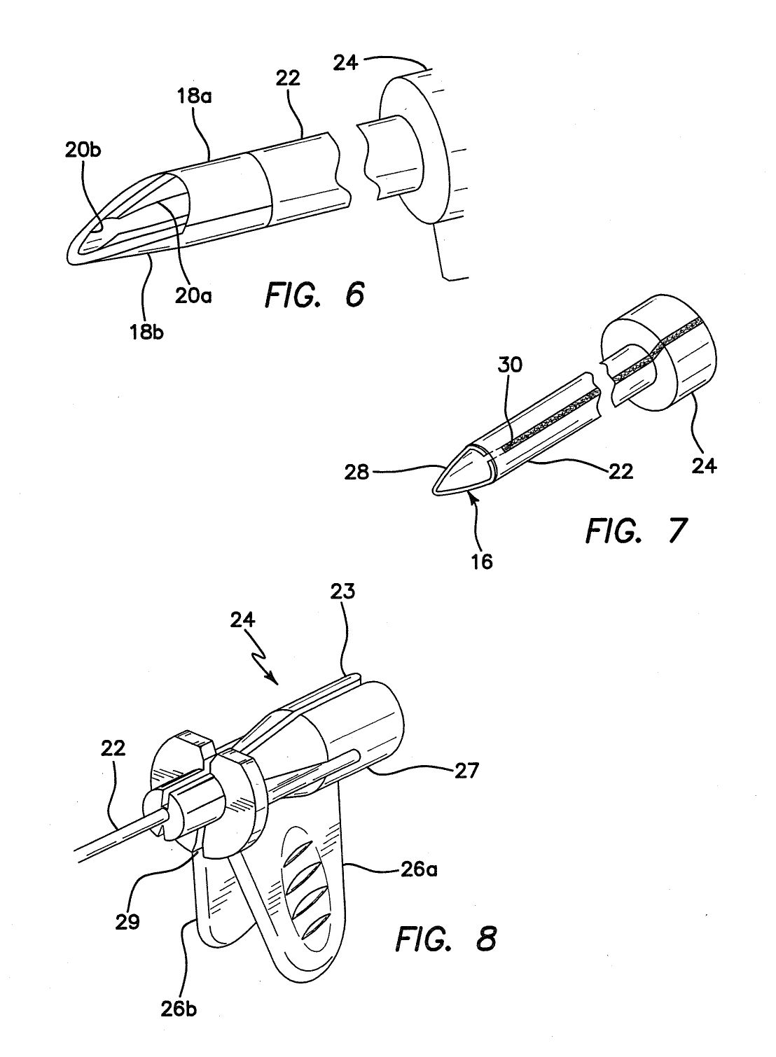

Another embodiment for the needle 16 as shown in FIG. 6 is comprised of a coaxial pair of cannula 18a and 18b, each with longitudinal slots 20a and 20b (i.e; a 23 gauge extra thin wall (0.019″ ID×0.025″ OD) both inside a single 21 gauge ultra thin walled sleeve 22 (0.027″ ID×0.032″ OD) again from K-Tube. Cannula 18a is coaxially disposed inside cannula 18b, A molded hub 24, which is coupled to the outer coaxial cannula 18b, holds the slotted cannula pair 18a and 18b so slots 20a and 20b are not aligned, and so that the tip bevels 26a and 26b of the cannula pair 18a and 18b are aligned as shown in FIG. 6. A lubricating coating between the two cannuli 18a and 18b (i.e silicone grease) keeps the two coaxial cannuli 18a and 18b sealed, but allows them to rotate relative to each other. The hub 24 is then selectively rotated to align the slots 20a and 20b in the two cannuli 18a and 18b so that the distal portion 12 of the guidewire 8 may be removed through the aligned slots 20a and 20b.

FIG. 7 is a perspective depiction in broken-away view showing a needle 16 and hub 24 having beveled distal end 28. Needle 16 and hub are tightly covered by an ultra thin walled sleeve 22, which serves to seal longitudinal slot 30 defined in needle 16 and hub 24. In the embodiment of FIG. 7 slot 30 is singular, i.e. there is no other slots extending the length of needle 16 and hub 24 at least for the purpose of allowing release or removal of the guidewire 8 disposed into the needle 16. In addition in the illustrated embodiment, slot 30 is continuous through needle 16 and hub 24, so that a guidewire 8 or other elongate element extending through needle 16 and hub 24 can be readily pulled through slot 30, tearing through sleeve 22 and completely removed from needle 16 and hub 24. Thus, slot 30 has a width which is at least wide enough to allow passage of the distal or narrower portion of the stepped guidewire 8 to pass through it. It must be understood that other topologies or path shapes may be chosen for slot 30 along the length of needle 16 and hub 24 if desired without departing from the scope of the invention. Further, while the preferred embodiment contemplates one or more tearable sleeves 22 which completely wrap around needle 16 and/or hub 24, the invention also contemplates a partial sleeve or sleeves 22 which may be adhered to needle 16 and/or hub 24 and wrap only partially around those elements. Similarly, it is further contemplated in another embodiment that slot 30 may be simply filled in or covered with a filler material, such as a polymer, or adhesive such as a UV acrylic adhesive from Loctite Henkel Corporation, Rocky Hill, Conn., which seals slot 30 and provides mechanical continuity and sealing during normal use, but which is still soft enough or shearable so that guidewire 8 can be easily manually pulled through it when it is desired to remove guidewire 8 from needle 16 and/or hub 24. Thus, the term “sleeve” is to be interpreted in this specification to include each of these embodiments and their equivalents.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

For example and by way of a summary, various illustrated embodiments of the splittable needle include:, 1) A splittable needle 16 through which the smaller 0.018 diameter portion 12 of the guidewire 8 is passed through the needle 16 into the vessel, the needle 16 is withdrawn, split apart, and removed allowing the larger 0.038 portion 10 of the guidewire 8 to be advanced. 2) An over-the-needle splittable catheter design, for which a splittable catheter (not shown) is placed with the needle 16, the needle 16 is withdrawn, and the catheter is used to deliver the tapered guidewire 8. 3) A co-axial slotted needle 16 constructed of two closely fitted cannula 18, each with a 0.020″ wide slot 20 down the length. The needle 16 is placed with the slots 180 degrees opposed, and the hub 24 sealed, allowing the unit to be placed like a traditional needle. The small diameter portion 12 of the guidewire 8 is placed into the vessel, and the needle hub 24 is rotated 180 degrees so that the longitudinal slots 20 are aligned. The needle 16 can then be removed from the small diameter portion 12 of the guidewire 8. 4) A slotted needle 16 with an attached tightly fitted thin wall coaxial sleeve 22. The guidewire 8 is placed through the needle 16 into the vessel, and the needle 16 is withdrawn such that the guidewire 8 tears the thin wall sleeve 22, allowing the needle 16 to be removed from the 0.018″ portion 12 of the guidewire 8. This embodiment allows the needle 16 to be removed with one hand while the guidewire 8 is being held with the other hand, in one continuous motion. There is some stress applied to the guidewire 8 as it stresses the thin wall sleeve 22 to tear, so a nitinol guidewire 8 may be required to prevent kinking of the guidewire 8. The preferred embodiment utilizes a 21G regular or thin walled needle 16 with a 0.020″ wide slot 20 and a 0.00025″ wall sleeve 22 that is shrunk down over the needle cannula 18.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

1. A method comprising:
inserting a micropuncture device into a body cavity to achieve a micropuncture into the cavity while reducing tissue trauma at a puncture site;
telescopically disposing a stepped guidewire having a proximal end and a reduced distal diameter portion for telescopic disposition through the inner diameter of the micropuncture device, and a proximal portion having a diameter larger than the inner diameter of the micropuncture device;
removing the micropuncture device from the guidewire while leaving the distal portion of the guidewire extending through the micropuncture and without removing the micropuncture device over the proximal end of the guidewire; and
advancing the proximal portion of the guidewire through the micropuncture into the body cavity.

2. The method of claim 1 further comprising guiding a large diameter instrument over the guidewire through the micropuncture using the proximal portion.

3. The method of claim 1 further comprising guiding a larger diameter instrument telescopically over the guidewire through the micropuncture using the distal portion as a guidewire only through the micropuncture.

4. The method of claim 1 where removing the micropuncture device comprises longitudinally separating the micropuncture device along a pair of score lines defined into the micropuncture device.

5. The method of claim 1 where the body cavity comprises a vascular cavity, and where inserting a micropuncture device into a body cavity comprises making a micropuncture into a vascular cavity.

6. The method of claim 1 where the micropuncture device comprises a microneedle and a sleeve telescopically disposed over the microneedle, further comprising removing the microneedle from the sleeve while retaining the sleeve in the body cavity, where telescopically disposing the stepped guidewire through the micropuncture device comprises telescopically disposing the stepped guidewire through the retained sleeve, where withdrawing the micropuncture device comprises withdrawing the microneedle from the sleeve, and where removing the micropuncture device comprises separating the sleeve without removing the sleeve over the proximal end of the guidewire.

7. The method of claim 1 where the micropuncture device comprises two slotted coaxial cannuli which are relatively rotatable with respect to each other and where removing the micropuncture device from the guidewire without removing the micropuncture device over the proximal end of the guidewire comprises rotating the two slotted coaxial cannuli with respect to each other to align a slot defined in each of the needles with each other, so that the guidewire is removed from the two cannuli through the aligned slots.

8. The method of claim 1 where the micropuncture device comprises a slotted needle with a separable sleeve coaxially disposed around the needle and where removing the micropuncture device from the guidewire without removing the micropuncture device over the proximal end of the guidewire comprises separating a portion of the sleeve covering the slot in the needle and removing the guidewire through a separation in the sleeve.

9. The method of claim 1 where the micropuncture device comprises a needle and a separable over-the-needle catheter, and where removing the micropuncture device from the guidewire without removing the micropuncture device over the proximal end of the guidewire comprises removing the needle from the catheter, and longitudinally separating the catheter to remove the guidewire through the separated catheter after telescopically disposing the stepped guidewire through the separable over-the-needle catheter.

10. The method of claim 1 where removing the micropuncture device from the guidewire without removing the micropuncture device over the proximal end of the guidewire comprises removing the guidewire through a separable sleeve covering a slot defined along the micropuncture device.

11. The method of claim 1 where the micropuncture device includes a hub and where removing the micropuncture device from the guidewire without removing the micropuncture device over the proximal end of the guidewire comprises removing the guidewire through a selectively openable slot defined in the hub.

12. The method of claim 1 where the micropuncture device includes a bifurcated hub and where removing the micropuncture device from the guidewire without removing the micropuncture device over the proximal end of the guidewire comprises removing the guidewire through a selectively openable slot defined in the bifurcated hub.

13. An apparatus comprising:
a longitudinally separable micropuncture device having an inner diameter for puncturing into a vascular cavity while reducing tissue trauma at a puncture site; and
a stepped or tapered guidewire having a proximal end and a distal portion capable of telescopic disposition through the inner diameter of the micropuncture device, and a proximal portion having a diameter larger than the inner diameter of the micropuncture device, the separable micropuncture device being configured to allow removal from the guidewire without removing the micropuncture device over the proximal end of the guidewire, the proximal portion of the guidewire being configured to guide a nonmicropuncture vascular instrument.

14. The apparatus of claim 13 where the distal portion of the guidewire is capable of telescopic disposition through the inner diameter of the micropuncture device and has a diameter of approximately 0.018-0.021 inch and where the proximal portion of the guidewire has a diameter larger than the inner diameter of the micropuncture device and is approximately 0.035-0.038 inch.

15. The apparatus of claim 13 where the distal portion of the guideware is characterized as a microguidewire having a diameter such that penetration of distal portion into tissue and disposition into a vascular cavity reduces tissue trauma.

16. The apparatus of claim 13 where the guidewire is in further combination with a larger diameter instrument and where the proximal portion of the guidewire is characterized as suitable in providing a guiding force to the larger diameter medical instrument which is telescopically disposed over the proximal portion of the guidewire.

17. The apparatus of claim 15 where the distal portion of the guidewire is chosen with a size of diameter so that it is flexible and atraumatic, but is not configured to be used to guide a larger medical instrument.

18. The apparatus of claim 16 where the proximal portion of the guidewire has a diameter such that it guides a larger medical instrument and can be practically steered in the vascular system.

19. The apparatus of claim 14 where the distal portion of the guidewire is preferably about 30 cm long and the length of the proximal portion of the guidewire is preferably about 30 cm long.

20. The apparatus of claim 13 where the transition between the proximal and distal portions of the guidewire is a sharp step.

21. The apparatus of claim 13 where the transition between the proximal and distal portions of the guidewire is a gradual taper whose longitudinal length is approximately ten diameters of the proximal portion of the guidewire.

22. The apparatus of claim 13 where the micropuncture device comprises a microneedle and a separable sleeve telescopically disposed over the microneedle, the microneedle being arranged and configured to be withdrawn from the sleeve while a portion of the sleeve is retained in the body cavity.

23. The apparatus of claim 13 where the micropuncture device comprises two slotted coaxial micro-cannuli which are relatively rotatable with respect to each other so that the guidewire is removable from the two micro-cannuli through the aligned slots.

24. The apparatus of claim 13 where the micropuncture device comprises a slotted microneedle with a separable sleeve coaxially disposed on the microneedle so that a portion of the sleeve covering the slot in the needle is separable and the guidewire is removable through a separation in the sleeve.

25. The apparatus of claim 13 where the micropuncture device comprises a microneedle and a separable over-the-needle catheter so that the guidewire is removable through the separated catheter after telescopically disposing the stepped guidewire through the separable over-the-needle catheter.

26. The apparatus of claim 13 where the micropuncture device is a microneedle with a single longitudinally extending slot defined therethrough with a width of the slot sufficient to allow withdrawal of the distal portion of the guidewire to pass therethrough and a separable sleeve covering the slot through which the distal portion of the guidewire can be pulled.

27. The apparatus of claim 26 further comprising a hub coupled to the microneedle, the slot defined through the hub and the separable sleeve covering the slot of the hub.

28. The apparatus of claim 13 further comprising a hub coupled to the micropuncture device and a selectively openable closed slot defined through the hub.

29. The apparatus of claim 28 where the hub is a bifurcated hub and where the selectively openable or closeable slot is defined in the hub.

30. An apparatus for use in introducing a non-micropuncture vascular device into a vascular system comprising:
separable means for producing a micropuncture into the vasculature while reducing tissue trauma at a puncture site;
first means for maintaining access through the puncture site into the vascular system while reducing tissue trauma, the first means for maintaining access being telescopically disposed within the separable means for producing the micropuncture; and
second means having a proximal end for guiding the medical instrument within the vascular system, the separable means being removable from the second means without removal over the proximal end of the second means, the second means being disposed into the vascular system through the micropuncture after the separable means for producing the micropuncture is removed from the first means for maintaining access.

31. The apparatus of claim 30 where the first means guides the non-micropuncture vascular device only through the puncture site.

32. The apparatus of claim 30 where the separable means for producing the micropuncture comprises a microneedle with at least two longitudinal score lines along which the microneedle can be manually separated.

33. The apparatus of claim 30 where the separable means for producing the micropuncture comprises a microneedle with a mounted separable sleeve.

34. The apparatus of claim 30 where the separable means comprises a microneedle with a single longitudinally extending slot covered with a separable sleeve.

35. The apparatus of claim 30 where the separable means comprises a microneedle and a hub coupled to the microneedle, the microneedle and hub having a selectively openable sealed slot defined at least partially therethrough.

36. The apparatus of claim 35 where the hub comprises a bifurcated hub and where the slot is defined in the bifurcated hub.
